(19) United States
(12) Patent Application Publication
(10) Pub. No.: US 2008/0077054 A1
(43) Pub. Date: Mar. 27, 2008

(54) Cervical dilator and methods of use

(75) Inventors: Gerald Feuer, Atlanta, GA (US); Gerald J. Sanders, Sonoma, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: FEMSUITE, LLC, San Francisco, CA (US)

(21) Appl. No.: 11/511,912

(22) Filed: Aug. 28, 2006

(51) Int. Cl. A61B 5/103

(52) U.S. Cl. 600/591

(57) ABSTRACT

In one embodiment, a cervical dilator includes a tubular member having a rounded cap disposed at the distal end, a balloon disposed on the distal portion, a lumen extending longitudinally within the tubular member, and a flexible wire attached to the wall of the lumen. The cervical dilator may be extended inside the uterus of a patient for sounding, and the tubular member will bend but not snap if excessive pressure is applied against a uterine wall, sliding away from the point of contact. The balloon is then properly positioned inside the cervical canal and gradually inflated. In other embodiments, a plurality of tubular members may be included, each reciprocating within another, and one or two applicator members may also be provided, to retain the cervix in position before and after dilation.

### FIELD OF THE INVENTION

The present invention relates to cervical dilators for sounding the uterine cavity and for expanding the cervical canal. More particularly, the present invention relates to cervical dilators that minimize the risk of uterine perforations during sounding and that provide for a gradual, atraumatic expansion of the cervical canal.

### BACKGROUND OF THE INVENTION

The cervix of a non-pregnant, reproductive age woman is a dense, yet distensible organ that has a resting diameter of 3-4 mm. Gynecological and obstetrical procedures often require a dilation of the cervix, to inspect visually the cervical and uterine regions and to access the uterine cavity with different types of medical instruments.

The cervix ordinarily responds to outward pressure by expanding within anatomical limits, but such pressure must be applied gradually to minimize damage. When pressure is continuously applied for even a short period of time, the cervix will remain temporarily in the dilated condition even after the pressure has been removed.

Cervical dilation is typically performed by employing a series of tapered rods called dilators. During a dilation procedure, a clinician initially stabilizes the cervix by using a single tooth tenaculum, which is a scissors-like instrument having opposing spikes at the terminal ends. The clinician may also use the tenaculum to apply counter traction on the cervix while thicker dilating rods are successively inserted. The dilating rods are often inserted by the clinician into the uterine cavity until contact with a uterine wall is made, so that the clinician may “sound” the uterus, that is, may determine size and position of the uterus within the abdomen of the patient.

While this dilation procedure is relatively simple, complications are common. Because the interior of the cervical canal cannot be visualized while the dilators are being inserted, the clinician must depend entirely on her expertise and sense of touch to guide the dilators into the cervical canal and then into the uterine cavity, and to know when pressure on the dilator should be released before a uterine wall is perforated.

During the majority of dilation procedures, the dilators can be inserted into the cervical canal with relative ease, and the uterine wall is firm enough to be felt by an experienced clinician upon contact. In a number of women, however, particularly in post-menopausal women and in women that have undergone surgeries as a consequence of a cervical disease, the cervix may be less elastic because of natural age-related hardening or because of scars. Consequently, the clinician must apply greater pressure on the dilator rods, both causing the tenaculum to tear through the tissue and lacerate the cervix, and making it difficult for the clinician to assess whether the lesser resistance encountered at the end of the procedure is caused by the successful dilation of the cervix or by a perforation of the uterine wall. It is estimated that approximately five percent of all cervical dilations result in uterine perforations.

Fortunately, most uterine perforations heal without any permanent ill effect. In fact, before hysteroscopy, nearly all perforations were merely suspected rather than known with certainty, because the procedure was completely blind and the clinician would suddenly notice no resistance when the tip of the dilator had reached the point where it should have been stopped by the uterine wall.

As the hysteroscope became employed with greater frequency during routine gynecological practice, perforations became both more likely and more dangerous. The hysteroscope is a long, narrow telescopic tool that is connected to a light source and that illuminates the area to be visualized. The distal end of the telescope is inserted through the dilated cervical canal, and, under direct visualization, the instrument is advanced into the uterine cavity. A camera is commonly attached to the proximal end of the hysteroscope to broadcast the image onto a large video screen.

Hysteroscopy requires a greater degree of dilatation than simple curettage, and also requires the injection of pressurized fluids into the uterine cavity. Accordingly, the procedure must be terminated as soon as a uterine perforation is detected, or large volumes of fluids, as well as any tissue surgically removed, might be washed directly into the peritoneal cavity.

At the same time, as laparoscopic and other abdomen-related procedures have also become more common, it has become increasingly important to “sound” the uterus, that is, to determine size and position of the uterus before laparoscopic or gynecological procedures are performed. Uterine sounding is often performed contemporaneously with cervical dilation and requires the insertion of a probe inside the uterus until the fondus of the uterus is felt, further increasing the risk of uterine perforations.

Apparatus for cervical dilation that do not require the use of tapered rods have been proposed in the prior art. For example, U.S. Pat. No. 3,848,602 to Gutnick discloses apparatus and processes for cervical dilation that include a balloon and an anchoring member at the distal end, each connected to a separate lumen. Gutnick's invention, however, does not resolve the problem of uterine perforations and also provides no direct measurements of cervical dilation, which may lead to an excessive expansion of the cervical canal and to related damages. Additionally, Gutnick's invention includes components and structures that appear to be particularly suited for pregnancy terminations rather than for general gynecological and obstetrical use.

Expansion devices using multiple balloons have also been proposed in the prior art. For example, U.S. Pat. No. 4,664,114 to Ghodsian teaches a rod having a rounded tip that decreases pain related to penetration through the unexpanded cervical canal and that also has two balloons disposed distally on the rod. The most distal balloon engages the inner os of the cervix, providing an anchor for maintaining the device in proper position while the second balloon is expanded inside the cervical canal. A disc and a coil spring are also provided proximally of the second balloon to prevent excessive penetration of the device into the uterine cavity. Ghodsian's invention still does not resolve the problem of avoiding perforations during the sounding of the uterus, and requires a multi-step dilation process based on the deployment of multiple balloons. Additionally, the presence of the disk occludes the field of vision of the clinician.

U.S. Pat. No. 4,693,704 to Ogita also discloses a catheter carrying two independent balloons, or alternatively a single, gourd shaped balloon. A plurality of apertures in different positions of the catheter provide for the delivery of medications to the cervix or to the uterus, and for the delivery of an inflating fluid to the balloon(s). Ogita's invention still does not solve the problem of avoiding uterine perforations and of obtaining a uniform and measurable cervical expansion, because it appears to be structured and operated for preventing a premature birth in pregnant women, rather than for sounding the uterus and for expanding the cervix in a gradual and uniform manner.

U.S. Pat. No. 4,976,692 and U.S. Published Application No. 2006/0058831, both to Atad, also disclose multi-balloon cervical catheters that require the use of two balloons during the procedure and that still do not solve the problem of uterine perforations during sounding. It should be observed that Atad's devices are not constructed for sounding the uterus and expanding the cervical canal, but for delivering certain types of medications to the cervical regions while the two balloons seal the ends of the cervical canal.

Another multi-balloon cervical catheter is disclosed in U.S. Pat. No. 5,104,377 to Levine. A first balloon is positioned in the uterus and a second balloon is partially inserted through the cervical canal, causing the two balloons to exert a clamping force towards each other and securing a shaft inside the cervical canal that connects the two balloons. Levine's catheter not only is limited by its inability to dilate the cervical opening beyond the diameter of the shaft, but also does not resolve the problem of perforations of the uterine wall during sounding.

A cervical catheter carrying a single balloon is disclosed in U.S. Pat. No. 5,338,297 to Kocur et al. In Kocur's device, a threaded metal tube has a balloon disposed in its distal portion that is snugly fit into the cervical canal, while a ring is positioned against the cervical os through a threaded connection with the tube. A medication is then dispensed into the uterus through a lumen in the metal tube and is prevented from flowing out of the cervical canal by the balloon-ring arrangement. It is apparent that Kocur's invention is not aimed at achieving a gradual expansion of the cervical canal nor to a safe sounding of the uterus. Kocur's invention also requires metal components that are possibly very harsh on any uterine surface, and does not provide observation of the cervical region by visual contact due to the use of a ring positioned against the cervix.

U.S. Pat. No. 5,947,991 to Cowan also discloses a catheter having a single balloon, which includes distal and proximal portions that have larger diameters than the narrower middle portion. The shape of the balloon in Cowan's device is aimed at stabilizing the cervix during expansion, but it is questionable whether a uniform expansion of the cervical canal can be achieved with this device, or how effectively the clinician could effectively see the cervix while the proximal end of the balloon expands and covers the cervix. Cowan's invention is intended for ripening the cervix prior to the onset of labor and provides no sounding capabilities. Cowan's invention also provides no protections against perforations if the uninflated device were employed for sounding the uterus of a non-pregnant woman.

A single balloon cervical catheter is disclosed in U.S. Published Application No. 2002/0111602 to Ackerman et al., which concerns a catheter for sealing the inner and outer os of the cervix, in order to prevent a loss of the medication dispensed from the catheter. The balloon in Ackerman's device has proximal and distal ends that expand outwards of the cervix to improve the sealing effect, but that also reduce or eliminate visual contact of the clinician. Ackerman's device also requires a translating inflation sleeve applied to the catheter and provides no uterine sounding capability.

U.S. Published Applications 2004/0116955, 2005/0055043 and 2005/0149100 to Foltz et al. are still directed to cervical canal dilators having two balloons, of which one is disposed in the uterus and the other in the cervical canal. The two balloons are contained within a retractable sheath, and the balloon disposed in the cervical canal is limited in its ability to expand diametrally. A stiffening wire may be added to provide added rigidity to the dilator. Therefore, the dilators in Foltz' inventions have limited dilating capabilities, and still provide no solutions to the problem of uterine perforations.

A cervical dilator with two expandable balloons is also disclosed in U.S. Application Publication No. 2004/0122463 to Hibler. One of the two balloons is used for an initial expansion of the cervical canal, and the other balloon is used for a successive expansion of the cervical canal. Hibler's invention, therefore, requires multiple balloons to perform different degrees of expansion, provides no sounding capabilities, and still does not address the problem of uterine perforations.

A cervical dilator is described in U.S. Application Publication 2004/0127931 to Kincaid et al., and includes a balloon at the distal end of the dilator that is inflated by releasing compressed gas into a piston, and by having the compressed gas drive an inflating fluid into the balloon. A rigid wire inside the balloon provides the balloon with the desired shape and angle. Besides offering no sounding features, Kincaid's device appears to be of complicated construction, requiring a gun-like structure to eject the compressed gas from its container.

Therefore, it would be desirable to provide a cervical dilator that enables a clinician to precisely control the dilatation of the cervix.

It also would be desirable to provide apparatus and methods for dilating the female cervix while minimizing the possibility of injury or harm to the patient.

It further would be desirable to provide apparatus and methods for safely sounding the uterine cavity prior to dilating the cervical canal.

It still further would be desirable to provide a device that is intuitive and easy to use by the medical practitioner.

It yet further would be desirable to improve the visibility and placement of an inflatable cervical dilator in its intended environment.

### SUMMARY OF THE INVENTION

In view of the foregoing, it is an object of the present invention to provide apparatus and methods for dilating the female cervix which reduce the possibility of injury or harm to the patient.

It is another object of the present invention to provide apparatus and methods for safely sounding the uterine cavity prior to dilating the cervical canal.

It is also an object of the present invention to provide a cervical dilator that enables a clinician to precisely control the dilatation of the cervix.

It is a further object of the present invention to reduce the need for considerable skill by the clinician during a cervical dilatation and to improve the accuracy of placement of the device in the intended environment.

It is a still further object of the present invention to provide apparatus for cervical dilation that are intuitive and easy to use by the medical practitioner.

It is a yet further object of the present invention to provide apparatus that can not only dilate the female cervix, but that also retain the cervix in an open condition after the dilator is removed.

It is another object of the present invention to provide such benefits in apparatus for cervical dilation that can be manufactured at low cost and that are disposable.

These and other objects of the present invention are accomplished by providing apparatus that can safely perform both uterine sounding and cervical dilation. The apparatus of the present invention provide the clinician with visual control of the procedure, with a uterine sounding that minimizes perforation risks, and with a graduated and controlled dilation of the cervical canal, while also providing the patient with a less traumatic intervention than in the prior art.

In a first embodiment, a cervical dilator includes a tubular member having a rounded cap at the distal end and a balloon disposed in the distal portion of the tubular member. An injection device is attached to the proximal end of the tubular member and is in fluid communication with the balloon through a lumen extending longitudinally in the tubular member.

The cervical dilator is first inserted in the cervical canal and is then extended into the uterine cavity. This enables the clinician to sound the uterus, and, in the event of an excessively forceful contact with a uterine wall, a flexible wire attached to the wall of the lumen causes the tubular member to arch but not snap, and at the same time the shape of the rounded cap makes the distal end of cervical dilator slide along the uterine wall without perforating it.

After sounding, the balloon is adjusted in the cervical canal and progressively inflated, causing the cervical canal to gradually expand, typically in one millimeter increments. The balloon has a central portion, that is essentially cylindrical, and tapered end portions. A colored marking disposed at the transition between the cylindrical portion and the proximal tapered end portion of the balloon enables the clinician to position the proximal tapered end portion to extend out of the cervical os and into the vaginal canal, preventing the cervical os from applying inward pressure on the balloon.

In a second embodiment of the invention, a cervical dilator includes an outer tubular member having a longitudinal lumen and a balloon disposed on its distal portion. A second longitudinal lumen provides fluid communication between an injection device and the balloon. An inner member reciprocates within the lumen of the outer tubular member and carries a rounded cap at its distal end. A flexible wire is disposed longitudinally within the inner member.

The clinician first sounds the uterine cavity by inserting the outer tubular member in the cervical canal and by extending the inner member into the uterus until contact with the uterine wall is made. As in the first embodiment, the risk of uterine perforations is minimized due to the construction of the inner member, in particular, due to the rounded cap disposed at the distal end and to the flexible wire disposed within.

In other embodiments, the cervical dilator also includes an applicator member, that is inserted into the cervical canal after dilation to maintain the cervix in a dilated condition. The applicator member preferably includes one or more grooves or protrusions on its outer surface, giving the applicator member a screw-like configuration and generating a stronger anchoring of the applicator member to the cervical os.

In these other embodiments, the cervical dilator has a tubular member with a larger diameter in the proximal portion and a narrower diameter in the distal portion. The applicator member is detachably connected to the proximal portion of the tubular member, while the balloon is disposed in the distal portion of the tubular member. After sounding the uterus and dilating the cervical canal, the balloon is deflated and the applicator member is moved forward and anchored to the cervix. The tubular member is then detached from the applicator member and extracted from the patient by sliding it through the lumen of the applicator member, which, once unobstructed, provides access to the uterine cavity. Alternatively, two tubular members may be provided, one reciprocating within the other, with the outer tubular member carrying the applicator member and with both tubular members sliding through the applicator member after anchoring to the cervix.

In still other embodiments, two applicator members are provided, one that is anchored to the cervical os prior to dilation, to retain the cervical canal in position, and another one that is anchored to the cervical os after dilation, to maintain the cervical canal in dilated condition, providing access to the uterine cavity for any necessary procedures.

Methods of use of the cervical dilators of the present invention are also described.

### BRIEF DESCRIPTION OF THE DRAWINGS

The above and other objects and advantages of the present invention will be apparent upon consideration of the following detailed description, taken in conjunction with the accompanying drawings, in which like reference numerals refer to like parts throughout, and in which:

FIG. 1 is a front view of a cervical dilator having a single tubular member, in accordance with a first embodiment of the invention;

FIG. 2 is a schematic view of the cervical dilator of FIG. 1 inserted into a cervical canal;

FIG. 3 is a cross-section of the tubular member of the dilator of FIG. 1;

FIG. 4 is a front view of a cervical dilator having an outer tubular member and an inner reciprocating member, in accordance with a second embodiment of the invention;

FIGS. 5A-5B are cross-sections of two variants of the inner tubular member of the embodiment of FIG. 4;

FIG. 6 is a front view of a cervical dilator inclusive of an applicator member, in accordance with a third embodiment of the invention;

FIG. 7 is a front view of a cervical dilator inclusive of an applicator member, in accordance with a fourth embodiment of the invention;

FIG. 8 is a front view of a cervical dilator inclusive of two applicator members, in accordance with a fifth embodiment of the invention; and

FIG. 9 is a front view of a cervical dilator also inclusive of two applicator members, in accordance with a sixth embodiment of the invention.

### DETAILED DESCRIPTION OF THE INVENTION

The present invention is related to apparatus that operate both as a uterine sounding devices and cervical dilation devices. The apparatus of the present invention provide the clinician with visual control of the procedure, with a uterine sounding that is essentially free of perforation risks, and with graduated and controlled dilation of the cervical canal. The inventive apparatus of the present invention also provide the patient with a less traumatic intervention than cervical dilators in the prior art.

In one embodiment, a cervical dilator according to the present invention includes a tubular member having a rounded cap disposed at the distal end and a balloon disposed on its distal portion. A clinician inserts the cervical dilator into the cervical canal of a patient with the balloon in a deflated condition and first sounds the uterus by extending the dilator into the uterus until the rounded cap contacts a uterine wall. A flexible wire, attached to the wall of the lumen, causes the distal portion of the tubular member to bend without snapping if excessive pressure is applied against the uterine wall, allowing the rounded tip to slide away from the contact point and preventing a perforation of the uterine wall. The balloon is successively positioned into the cervical opening and is gradually inflated, dilating the cervical canal and providing the clinician with access to the uterus for performing any necessary gynecological procedures.

The cervical dilator may also include one or two applicator members that are anchored to the cervical os and that operates like tenacula.

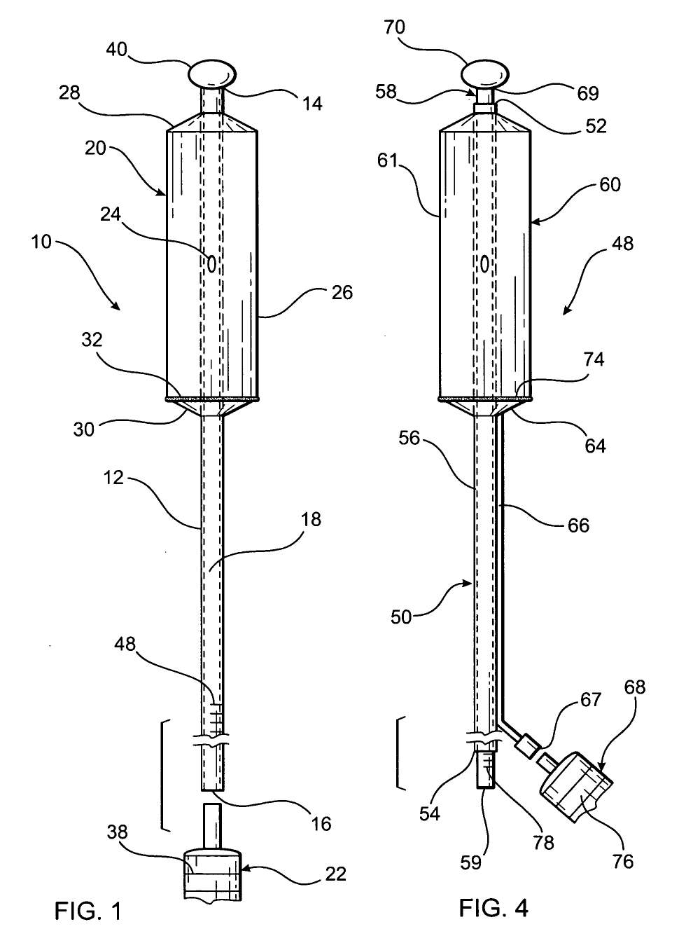

Referring first to FIG. 1, a first embodiment of a cervical dilator constructed in accordance with the principles of the present invention is described. In its most basic configuration, cervical dilator 10 includes tubular member 12, balloon 20 and rounded cap 40.

More particularly, tubular member 12 has a hollow, elongated shape that includes distal end 14, proximal end 16, and lumen 18 extending therebetween. Balloon 20 is disposed at the distal portion of tubular member 12 and is also in fluid communication with proximal end 16. When injection device 22 (for example, a syringe) is connected to proximal end 16, a fluid, either liquid or gaseous, can be transmitted from injection device 22 to balloon 20 through lumen 18. Aperture 24 in lumen 18 provides fluid communication between lumen 18 and the inner part of balloon 20, enabling the inflation of balloon 20. In a variant of the present embodiment, lumen 18 does not extend from proximal end 16 to distal end 14, but extends instead only from proximal end 16 to aperture 24.

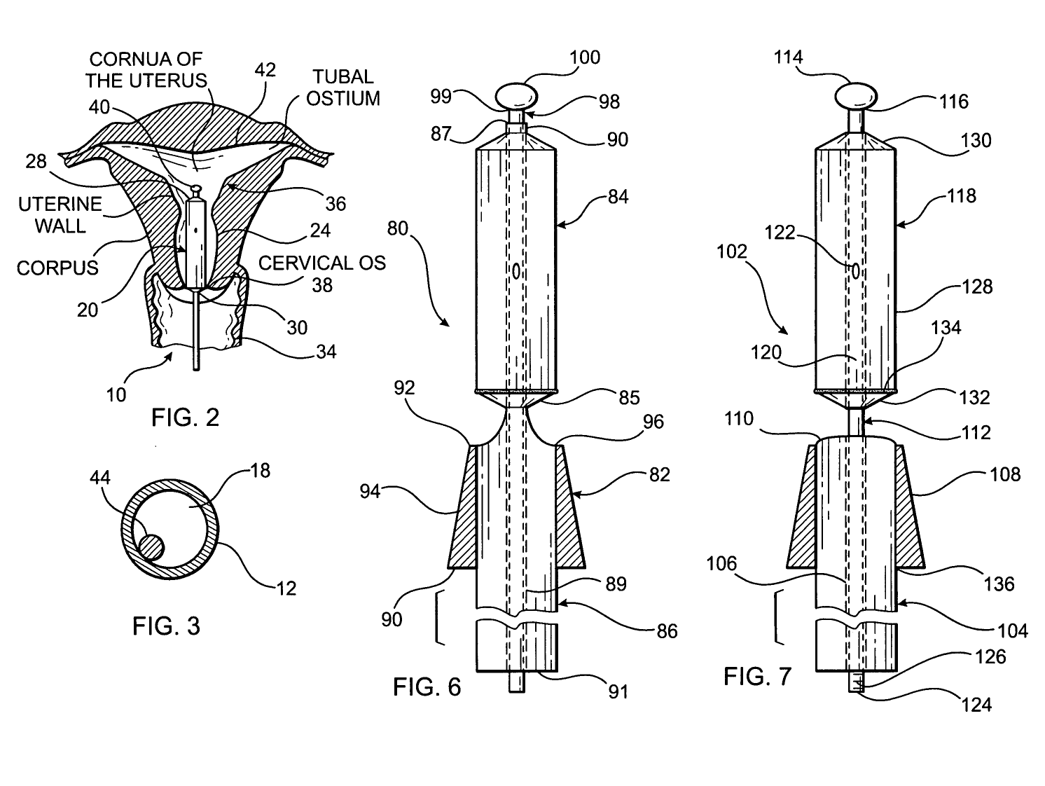

Referring now to FIG. 2, balloon 20 is adapted to be received in cervical canal 24, and has a size capable of effectively dilating cervical canal 24, typically a length of 6-8 cm and a width of approximately 1.5 cm. Balloon is preferably manufactured from a non-compliant material, so that its shape and dimensions when fully inflated are predetermined. Typically, balloon 20 has a shape that is essentially cylindrical and that includes cylindrical portion 26 and tapered end portions 28 and 30 disposed respectively in distal and proximal positions in relation to cylindrical portion 26. The essentially cylindrical configuration of balloon 20 provides for a uniform dilation of cervical canal 24, but one skilled in the art will recognize that other shapes of balloon 20 may be effectively employed to dilate cervical canal 24, for example, a shape that includes a central convex portion and two opposing tapered end portions that have side walls with different radii of curvature than the central convex portion.

Colored marking 32 is disposed at the transition line between cylindrical portion 26 and proximal tapered end 30, so that, when balloon 20 is inserted and later inflated into cervical canal 24, the operating clinician can position balloon 22 to have proximal tapered end portion 30 extending out of cervical canal 24 and into vaginal canal 34. This position of proximal tapered end portion 30 prevents balloon 20 from being drawn into uterine cavity 36 by the natural constrictive activity of cervical canal 24 and by the inward pressure applied by cervical os 36.

Balloon 20 may be inflated with air or with another gas, but is preferably inflated with a liquid saline solution. The saline solution (or any other desired fluid) is injected into balloon 20 from injection device 22, which is connected to lumen 18 at proximal end 16, for example, with a Luer fitting. One example of an injection device that may be employed is a syringe, which is readily available during medical procedures.

Balloon 20 is preferably inflated in diametral increments of one millimeter at a time, in order to cause cervical canal 24 to dilate in controlled incremental steps and to minimize cracks in the cervical os and shock to the patient. To assist the clinician in determining the most appropriate rate of injection of the saline solution into balloon 20, injection device 22 may carry a plurality of markings 38 on its outer surface, each corresponding to the amount of fluid required to be injected into balloon 20 to increase balloon diameter by one millimeter over the previous diameter.

Tubular member 12 is manufactured from a semi-rigid material, which flexes under longitudinal pressure but which will also eventually buckle and snap when the pressure on tube 12 exceeds a predetermined amount. One such material is surgical-grade polypropylene, but one skilled in the art will recognize that other materials may be advantageously employed for the same application, for example, high density polyethylene and polyester.

Rounded cap 40 is disposed at distal end 14 and, together with tubular member 12, acts as a sounding device. After the introduction of dilator 10 into cervical canal 24, the clinician extends dilator 10 further into uterine cavity 36 to “sound” the uterus, that is, to assess the depth and position of uterine cavity 36. Rounded cap 40 is preferably shaped like a sphere or an ellipsoid of a diameter larger than tubular member 12, reducing the risk of perforation of uterine wall 42 in the event the clinician applies an excessively forceful pressure, because the relatively large diameter of rounded cap 40 will makes perforation unlikely. On the contrary, a sharper and/or smaller distal end 14 disposed on tubular member 12 would provide distal end 14 with needle-like properties, significantly increasing the risk of perforation of uterine wall 42.

Referring now to FIG. 3, flexible wire 44 is disposed within lumen 18 to further reduce the risk of perforation of uterine wall 42 from rounded cap 40. Flexible wire 44 is attached to the inner wall of lumen 18, preferably by adhesive bonding, in order to create uniformity of movement and deformation between flexible wire 44 and tubular member 12. While the term “wire” is often indicative of a metal wire, one skilled in the art will appreciate that non-metallic wires may also be employed satisfactorily, for example, nylon wires or wires made from oriented plastics.

Flexible wire 44, disposed within lumen 18, prevents the snapping and single point buckling of tubular member 12 when a longitudinal pressure is applied on rounded cap 40 that is higher than a predetermined amount. The joining of flexible wire 44 with lumen 18 causes tubular member 12 to arch and then assume a rounded curvature (instead of a single point bending) when the pressure on rounded cap 40 exceeds the predetermined level. Clinical tests have shown that, upon application of longitudinal pressure on cervical dilator 10 against uterine wall 42, tubular member 12 arches, causing rounded cap 40 to slide away from the pressure point and preventing a perforation of uterine wall 42.

Tubular member 12 may optionally include markings 46 disposed on its outer surface, which provide the clinician with a visual indication of the depth of penetration of tubular member 12 inside uterine cavity 36, and, therefore of the size of uterine cavity 36.

A method of use of cervical dilator 10 is described next. In preparation for the sounding and dilation procedure, a speculum is employed to expand vaginal canal 34 and to enable the clinician to achieve visual contact with cervical os 38.

Tubular member 12 is inserted through vaginal canal 34 and through cervical os 38. Tubular member 12 is further inserted into uterus 36, and, when rounded cap 40 eventually contacts uterine wall 42, the clinician is provided with an initial determination of the size and position of uterus 36. Markings 48 will assist the clinician in determining a measurement of the depth of uterus 36. If excessive pressure is employed by the clinician, tubular member 12 will not puncture uterine wall 42 or snap, but, as described above, tubular member 12 will instead progressively bend while rounded cap 40 will slide away along uterine wall 42.

After sounding uterus 36, balloon 20 is properly positioned inside cervical cavity 24, with proximal tapered end 30 extending out of cervical os 38, thereby preventing cervical os 38 from applying pressure on proximal tapered end 30 and from pushing balloon 20 inside uterus 36. Some back-and-forth adjustment may be required to achieve the proper positioning of balloon 20 within cervical os 38. Marking 32, positioned at the transition between cylindrical segment 20 and proximal tapered end 30, provides the clinician with a visual guidance in determining the proper positioning of balloon 20 inside cervical canal 34.

Balloon 20 is then inflated in progressive increments of one millimeter each, up to a desired diameter, typically 15 mm. Markings 38 on injection device 22 will guide the clinician in injecting the appropriate quantity of fluid into balloon 20 to achieve a one millimeter increase in diameter over the previous diameter.

After uterine canal 24 is dilated to the desired diameter, balloon 20 is deflated and dilator 10 is retracted, leaving cervical canal in a dilated position for the successive intervention of the clinician.

Referring now to FIG. 4, a second embodiment of a cervical dilator constructed according to the principles of the present invention is described. Cervical dilator 48 comprises outer tubular member 50 having first lumen 56 extending between distal end 52 and proximal end 54. Inner member 58 is positioned within first lumen 54 and is longer than outer member 50, reciprocating within and extending out of first lumen 54 when a longitudinal pressure is applied to proximal end 59 of inner member 58.

Balloon 60 is disposed on the distal portion of outer member 50, and has a shape adapted to be received in cervical canal 24. Balloon 60 is preferably manufactured from a non-compliant material and will expand to have a shape comprising central cylindrical portion 61, distal tapered end portion 62 and proximal end tapered portion 64. As for the previously described embodiment, one skilled in the art will recognize that balloon 60 may be manufactured to expand to different shapes, all suitable for expanding cervical canal 24.

Balloon 60 is inflated with a gaseous or liquid fluid, preferably with a liquid saline solution. Second lumen 66 connects balloon 60 to fluid port 67 and runs parallel to outer tubular member 50, extending laterally from outer member 50 in the proximity of proximal end 54. An injection device 68, from which fluid is injected into balloon 58, is connected to port 67, for example, with a Luer fitting, and is in fluid communication with balloon 58.

Inner member 58 has rounded cap 70 affixed to distal end 69, to minimize the risk of perforations of uterine wall 42 during the sounding of the uterus. The diameter of rounded cap 70 is larger than that of inner member 58, to avoid a needle effect upon contact with uterine wall 42. Inner member 50 is manufactured from a semi-rigid material (for example, surgical grade polypropylene) that is prone to arching under longitudinal pressure, but that will eventually snap and buckle in a single point when longitudinal pressure exceeds a predetermined level.

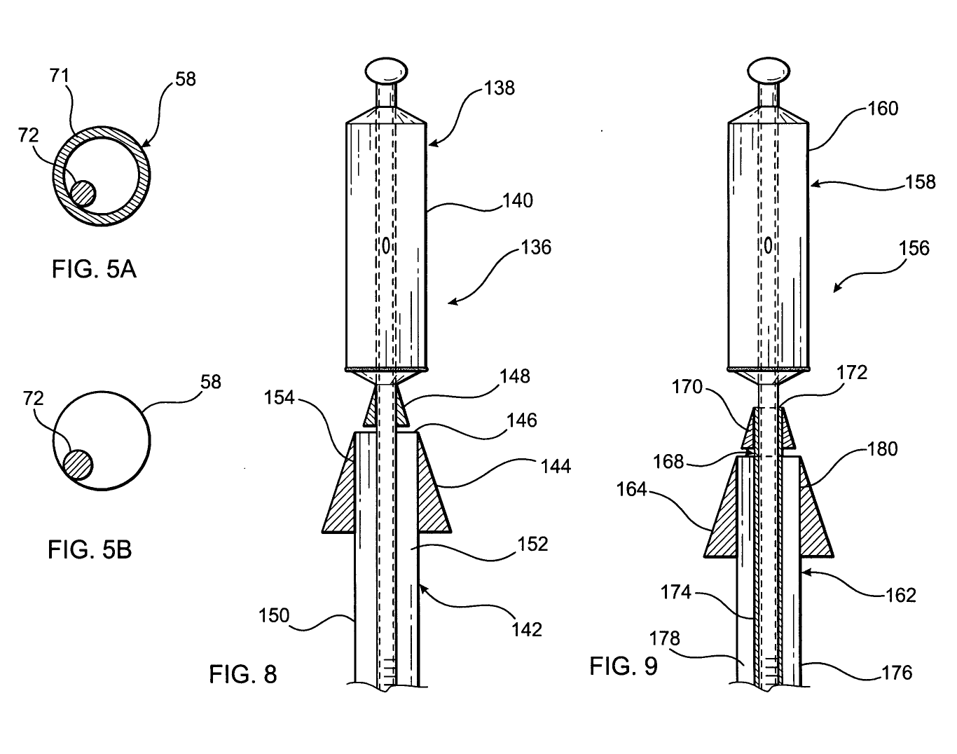

Referring further to FIGS. 5A-5B, inner member 58 includes flexible wire 79 that is embedded within inner member 58, preferably in an off-center position. If inner member 58 is tubular, that is, if inner member 50 has lumen 71 running longitudinally inside tubular member 58, flexible wire 72 is attached to the wall of lumen 71, as shown in FIG. 5A, moving and bending in the same manner as lumen 71. If inner member 58 is instead solid, flexible wire 72 is embedded within inner member 58 and may be disposed at the periphery of inner member 58, as shown in FIG. 5B.

Different markings may be provided on dilator 48, to facilitate use by a clinician. For example, colored marking 74 may be provided on balloon 58 at the transition line between central portion 60 and proximal tapered end 64, to provide visual guidance in extending proximal tapered end 64 out of the cervical os, thereby preventing cervical os 38 from applying an inward pressure on balloon 60 that would push balloon 60 into uterine cavity 36.

Markings 76 may also be disposed on injection device 68, to enable the clinician to inject an appropriate amount of fluid into balloon 60 that will cause an expansion of balloon 60 of one more millimeter over the previous diameter. Additionally, markings 78 may be disposed on inner member 58, to enable a clinician to measure the displacement of inner member 58 in relation of proximal end 54, and, consequently, the amount of penetration of inner member 58 into uterine cavity 36.

The method of use of cervical dilator 48 is described next. After vaginal canal 34 is dilated with a speculum and the clinician has established visual contact with cervical os 38, cervical dilator 48 is inserted into cervical canal 24, and inner member 58 is extended from outer tubular member 50 into uterine cavity 36. As in the previously described embodiment, the combination of rounded cap 70 and of flexible wire 72 causes inner member 58 to bend and slide along from uterine wall 42, in the event the clinician applies excessive pressure. The combined presence of rounded cap 70 and of flexible wire 72 significantly reduces the risk of uterine perforation.

Markings 78 disposed on inner member 58 enable the clinician to measure the depth of penetration of inner member 58 into uterus 36 and to have a rapid and relatively accurate measurement of the size of uterine cavity 36.

After the clinician has sounded the uterus, inner member 58 is retracted and balloon 60 is adjusted into cervical canal 24, with proximal tapered end portion 64 extending out of cervical os 38, which will prevent the inward pressure provided by cervical os 38 and by cervical canal 24 from pushing balloon 60 into uterine cavity 36. Advantageously, colored marking 74 provides the clinician with an indication of the position of proximal tapered end 64 in relation to cervical os 38.

Balloon 60 is then inflated up to a desired diameter, typically 15 mm. The inflation is performed one millimeter at a time, to prevent cracks on cervical os 38 and shock to the patient. Markings 76 on injection device 68 guide the clinician in determining the necessary amount of fluid to inject into balloon 60, in order to expand balloon 60 one millimeter over the previous diameter.

After cervical canal 24 has been expanded to the desired diameter, balloon 60 is deflated, and cervical dilator 48 is removed from the cervical canal 24 and vaginal canal 34.

Referring now to FIG. 6, a third embodiment of a cervical dilator constructed according to the principles of the present invention is described. Cervical dilator 80 has a structure that is similar to the previously described embodiments 10 and 48, but that further includes applicator member 82. The purpose of applicator member 82 is to enable the clinician to maintain cervical os 38 in a dilated position after balloon 84 has been deflated and retracted from cervical canal 24.

Cervical dilator 80 includes outer member 86 having proximal portion 88, that supports applicator member 82, and distal portion 90, that supports balloon 84. Longitudinal lumen 89 extends between distal end 87 and proximal end 89 of outer tubular member 86.

Applicator member 82 is preferably frustoconical in shape and includes major base 90, oriented proximally, minor base 92, oriented distally, and lateral wall 94. Lumen 96 extends longitudinally between major base 90 and minor base 92 of applicator member 82, and one or more grooves or protrusions are shaped on lateral wall 94, providing applicator member 82 with a screw-like external appearance.

Applicator member 82 is detachably connected to the proximal portion of outer member 86. For example, the proximal portion of outer member 86 and applicator member 82 may have a threaded connection, or protrusions extending from proximal portion 88 may lock with matching depressions carved within lumen 96. Additionally, applicator member 82 may be connected to a control arm (not shown) that enables the clinician to properly position applicator member 82 into cervical canal 24.

The diameter of proximal portion 88 of outer member 86 is larger than the diameter of distal portion 90, enabling the removal of outer member 86 from cervical canal 24 after dilation has been achieved, while applicator member 82 is inserted into cervical canal 24 to maintain the dilated condition. More specifically, after balloon 84 has been inflated and cervical canal 24 has been dilated, applicator member 82 is moved forward into cervical os 38 and twisted in place to achieve a stable anchoring. Proximal portion 88 is then detached from applicator member 82 and outer member 86, including now deflated balloon 84, is eventually slid through lumen 96 and removed from the patient, leaving instead applicator member 82 in place.

Cervical dilator 80 further includes inner member 98, which carries rounded cap 100 at distal end 99. Inner member 98 reciprocates within lumen 88 and has a flexible wire longitudinally embedded in its interior, causing inner member 98 to bend when longitudinal pressure exceeds a predetermined amount, rather than snap and buckle in a single point, in the same manner as in embodiment 48. A secondary lumen (not shown) or a tube mounted on inner member 98 (also not shown) carries an inflating fluid to balloon 84.

The method of use of cervical dilator 80 is similar to the method of use of cervical dilator 48, except for the use of applicator member 82. After vaginal canal 24 has been dilated with the aid of a speculum, cervical dilator 80 is introduced into cervical canal 24 and inner member 98 is further extended to sound uterine cavity 36. After the clinician has determined size and position of uterine cavity 36, balloon 84 is properly positioned within cervical canal 24, so that proximal tapered end portion 85 will extend out of cervical os 38. Balloon 84 is then inflated to the desired diameter in progressive increments, for example, in one millimeter increments, until the desired level of dilation of cervical canal 24 is achieved.

Balloon 84 is then deflated, and applicator member 82 is inserted into dilated cervical os 38. If grooves or ridges are present on outer surface 94, applicator member 82 is secured to cervical canal 24 through a turning motion, in a screw-like fashion. This maintains applicator member 82 anchored to cervical canal 24 while cervical canal 24 is in a dilated condition, in the same manner as a tenaculum. Outer member 86 is finally detached from applicator member 82, leaving applicator 82 in place while lumen 96 provides an entry passage to uterine cavity 36 to perform any necessary gynecological procedures.

One skilled in the art will appreciate that the steps of the presently described method may be performed in different orders, without altering the scope of the invention. For example, outer member 86 may be detached from applicator member 82 prior to anchoring applicator member 82 in cervical canal 24.

Referring now to FIG. 7, a fourth embodiment of a cervical dilator constructed according to the principles of the present invention is described. The basic structure of cervical dilator 102 includes outer tubular member 104, having lumen 106 extending longitudinally within outer tubular member 104, and applicator member 108 disposed in the proximity of distal end 110. Inner tubular member 112, having rounded cap 114 disposed at distal end 116 and balloon 118 disposed on its distal portion, reciprocates within lumen 106 and is longer than outer tubular member 104, extending out of outer tubular member 104. Distal end 110 of outer tubular member 104 is preferably rounded, in order to minimize contact of any sharp edges against of cervical os 38.

Inner tubular member 112 also includes lumen 120 extending longitudinally between distal end 116 and proximal end 124, so that a fluid (for example, a saline solution) can be injected into balloon 118 through one or more apertures 122 on lumen 120. An injection device (not shown) can be attached to proximal end 124 of inner tubular member 112 and is connected to lumen 120.

As in the previous embodiments, inner tubular member 112 includes a flexible wire attached to the wall of lumen 120, to provide for an arching deflection (instead of a snapping, single point deflection) of inner tubular member 112 when rounded tip 110 is pushed against a surface with a pressure higher than a predetermined level. Also as in the previous embodiments, balloon 118 is preferably made of a non-compliant material and inflates to define an essentially cylindrical central portion 128 and two tapered end portions, namely, distal tapered end portion 130 and proximal tapered end portion 132. A colored marking 134 on balloon 118 highlights the transition line between central portion 128 and proximal tapered portion 134.

Applicator member 108 is detachably connected to outer tubular member 104, for example, with a threaded connection. One skilled in the art will appreciate that a variety of other detachable connections may be employed to join outer tubular member 104 with applicator member 108. As in the previously described embodiment 80, applicator member 108 preferably carries one or more grooves or protrusions on its outer surface, providing applicator member 108 with a screw-like shape that enables a secure anchoring of applicator member 108 into cervical canal 24. Additionally, applicator member 108 may be connected to a control arm (not shown) that enables the clinician to properly position applicator member 108 into cervical canal 24.

A method of use of a cervical dilator 102 is described next. After vaginal canal 34 is expanded with a speculum, cervical dilator 102 is inserted into vaginal canal 34 and inner tubular member 112, carrying balloon 118, is extended into cervical canal 24. At this stage, outer tubular member 104 and applicator member 108 remain outside of cervical canal 24, because cervical canal 24 is still in an undilated state, making the diameters of outer tubular member 104 and of applicator member 108 excessively large for passage.

Inner tubular member 112 is further extended into uterine cavity 36 until contact with uterine wall 42 is achieved, providing the clinician with a sounding of uterus 36. As in the previously described embodiments, the construction of inner tubular member 112, which includes rounded cap 114 and the flexible wire disposed within lumen 120, minimizes the risk of puncturing uterine wall 42 when excessive longitudinal pressure is applied against uterine wall 42.

Markings 126, disposed on the proximal portion of inner tubular member 112, provide the clinician with a numeric measurements (for example, in centimeters) of the depth of penetration of inner tubular member 112 within uterine cavity 36.

After the sounding of uterine cavity 36 is completed, inner tubular member 112 is retracted and balloon 118 is properly positioned inside cervical cavity 24, with proximal tapered end portion 132 extending out of cervical os 38. Some back-and-forth adjustment may be required to achieve a proper positioning of balloon 112 within cervical os 38. Colored marking will assist the clinician in determining the proper positioning of balloon 112 within cervical canal 34.

Balloon 118 is then inflated in increments of one millimeter at a time, up to a desired diameter, typically 15 mm. After uterine canal 24 is dilated to the desired diameter, balloon 118 is deflated and outer tubular member 104 is advanced into cervical os 38. Applicator member 108 is then disposed into cervical canal 24 by twisting outer member 104 with a screw-like motion, and the grooves or protrusions on the outer surface of applicator member 108 will maintain applicator member 108 securely anchored to cervical canal 24.

Outer tubular member 104 is then disengaged from applicator member 108, by untwisting one from the other, if outer tubular member 104 and applicator member 108 are joined with a threaded connection. The assembly of outer tubular member 104 and inner tubular member 112, inclusive of balloon 118, is finally slid through lumen 126 of applicator member 108, leaving applicator member 108 to act as a tenaculum that maintains cervical os 38 in the dilated condition. The clinician is then provided with access to the uterine cavity through lumen 134, to perform any necessary gynecological procedures.

Referring now to FIG. 8, a fifth embodiment of a cervical dilator constructed according to the principles of the present invention is described.

Cervical dilator 136 comprises three basic elements, namely, tubular member 138, having balloon 140 disposed on its distal portion; outer member 142, having first applicator member 144 disposed at its distal end 146; and second applicator member 148, disposed on tubular member 138 proximally of balloon 140.

Tubular member 138 has the same construction as previously described cervical dilator 10 in the first embodiment, which will not be described again here for the sake of brevity. Second applicator member 148 is preferably frustoconical in shape and has the minor base oriented in the direction of balloon 140 and dimensions appropriate for fitting into undilated cervical canal 24. Second applicator member 148 may also carry one or more grooves or ridges spirally disposed on its outer surface, to provide second applicator member 148 with a screw-like configuration that facilitates anchoring of second applicator member 148 into cervical canal 24 prior to dilation.

Outer tube 150 supports first applicator member 144 on outer member 142 and has lumen 152 extending longitudinally along its length. The diameter of lumen 152 is sufficiently large to permit the passage of second applicator member 148 and balloon 140 in deflated condition.

First applicator member 144 also has a frustoconical shape, with the minor base oriented distally, and may carry one or more grooves or protrusions on its outer surface, to provide first applicator member 144 with a screw-like configuration. The dimensions of first applicator member 144 are appropriate for disposing first applicator member 144 within cervical os 38 after dilation, becoming anchored to cervical os 38 because of its screw-like configuration.

First and second applicator members 144 and 148 are typically manufactured from a non-metallic material, such as a silicone material that can be injection molded and can be manufactured with a cost sufficiently low to make first and second applicator members 144 and 148 disposable after use.

First applicator member 144 is detachably connected to outer tube 150, with a type of connection known to those skilled in the art, for example, with a threaded connection or with a tooth and groove connection. In one variant of the present embodiment, second applicator member 148 is also detachably connected to tubular member 138, again with a type of connection known to those skilled in the art.

A method of use of embodiment 136 is described next. After dilating vaginal canal 34 of the patient, the clinician inserts balloon 140 into cervical canal 24, maintaining tubular member 138 in cervical canal 24 in a stable position by anchoring second applicator member 148 to the undilated cervical os 38. If second applicator member 148 is detachably connected to tubular member 138, the clinician may first sound uterine cavity 42 by detaching second applicator member 148 from tubular member 138 and extending tubular member 138 into uterine cavity 42, prior to reattaching second applicator member 148 to tubular member 138 and to proceeding with cervical dilation.

After balloon 140 has been inflated and cervical canal 24 has been dilated, second applicator member 148 becomes disengaged from cervical os 38, because the outer diameter of second applicator member 148 is smaller than the diameter of cervical os 38 after dilation. Outer member 142, inclusive of first applicator member 144, is then advanced in cervical os 38, and first applicator member 144 is anchored to cervical canal 24. In one variant of the present embodiment, a control arm (not shown) is connected to first applicator member 144, to facilitate positioning into cervical os 38.

Tubular member 138 is then slid through lumen 152, removing it from the patient, and outer tube 150 is finally detached from first applicator member 144, leaving first applicator member 144 to operate as a cervical tenaculum that maintains cervical canal 24 in dilated condition that provides access to uterine cavity 42 through its lumen 154 for any necessary procedures.

In an alternative method of use, tubular member 138 may be removed from the patient prior to anchoring first applicator member 144 in cervical os 38 after dilation.

Referring now to FIG. 9, a sixth embodiment of a cervical dilator constructed according to the principles of the present invention is described. Cervical dilator 156 includes three basic elements, namely, tubular member 158, inclusive of balloon 160; outer member 162, having first applicator member 164 disposed at its distal end 166; and inner member 168, disposed between tubular member 158 and outer member 162. Second applicator member 170 is disposed at distal end 172 of inner member 168.

The construction of tubular member 158 is the same as that of cervical dilator 10 in the first embodiment, and will not be repeated here for the sake of brevity. The construction of outer member 162 is the same as that of outer member 142 in the previously-described embodiment, and will also not be repeated here for the sake of brevity.

Inner member 168 comprises inner tube 174, which reciprocates within outer tube 176 and within which tubular member 158 reciprocates, providing cervical dilator 156 with a three-tube construction. Second applicator member 170 is preferably frustoconical in shape and has one or more grooves or ridges on its outer surface, in the same manner as second applicator member 148 in the previously-described embodiment. The diameter of lumen 178 extending longitudinally within outer tube 176 is sufficiently large to enable the passage of second applicator member 170 and of balloon 160 (in deflated condition) through lumen 178.

The method of use of cervical dilator 156 is described next. After expanding vaginal canal 34, tubular member 158 is introduced into cervical canal 24 and is further extended into uterine cavity 42, to sound uterine cavity 42. Tubular member 158 is successively retracted to a position that places balloon 160 within cervical canal 24. Inner member 168 is then advanced into cervical os 38 and anchored to cervical os 38, maintaining cervical canal 24 in stable position while balloon 160 is inflated.

After cervical canal 24 has been dilated, second applicator member 170 becomes disengaged from cervical os 38, which, after dilation, has acquired a diameter larger than that of second applicator member 170. Inner member 168 and tubular member 158 (with the balloon in deflated condition) are then removed from the patient by sliding them through lumen 178, and outer member 162 is advanced to engage first applicator member 164 in cervical os 38. Alternatively, first applicator member 164 may be engaged in cervical os 38 before removing tubular member 158 and inner member 168.

After tubular member 158 and inner member 168 have been removed, and after first applicator member 164 has been anchored within cervical os 38, outer tube 176 is disengaged from first applicator member 164, leaving first applicator member 164 in position, so to maintain cervical canal 24 in dilated condition and to provide access to uterine cavity 42 through lumen 180 of first applicator member 164 for performing any successive clinical procedures.

While preferred embodiments of the invention are described above, it will be apparent to one skilled in the art that various changes and modifications may be made. The appended claims are intended to cover all such changes and modification that fall within the true spirit and scope of the invention.

What is claimed is:

1. A cervical dilator comprising:
a tubular member having a distal end, a proximal end, and a lumen therebetween, the tubular member being made of a semi-rigid material;
a rounded cap disposed at the distal end, the rounded cap having a diameter larger than the diameter of the tubular member;
a flexible wire extending inside the lumen from the cap to the proximal end, the flexible being attached to a lumen wall;
an elongated balloon disposed distally on the tubular member, the balloon having a size capable of dilating a cervical canal, the balloon being in fluid communication with the lumen.

2. The cervical dilator of claim 1, wherein the rounded cap is a sphere or an ellipsoid.

3. The cervical dilator of claim 1, wherein the balloon inflates to comprise a substantially cylindrical central portion and opposite proximal and distal tapered end portions.

4. The cervical dilator of claim 3, wherein a colored marking is disposed at the transition between the substantially cylindrical central portion and the proximal tapered end portion.

5. The cervical dilator of claim 1, further comprising an injection device connected to the proximal end of the tubular member and dimensioned to progressively inflate the balloon.

6. The cervical dilator of claim 5, wherein the injection device inflates the balloon with a saline solution.

7. The cervical dilator of claim 5, wherein the injection device carries markings corresponding to the quantities of saline solution to be injected to cause a progressive inflation of one millimeter of the substantially cylindrical central portion of the balloon.

8. The cervical dilator of claim 5, wherein the injection device is a syringe.

9. The cervical dilator of claim 1, wherein the tubular member carries markings on its outer surface, enabling a clinician to measure the depth of insertion of the tubular member into the cervical canal.

10. The cervical dilator of claim 1, further comprising an outer member having a proximal end and a distal end and a lumen therebetween, the tubular member reciprocating in the lumen of the outer member, a first applicator member being detachably disposed at the proximal end of the outer member, the first applicator member being dimensioned to fit within the cervical canal in dilated condition.

11. The cervical dilator of claim 10, wherein the first applicator member has a frustoconical body carrying one or more spirally disposed grooves or protrusions on its lateral surface, and wherein the first applicator member is detachably disposed on the outer member by detachably connecting the tubular member to a longitudinal lumen in the frustoconical body.

12. The cervical dilator of claim 10, further comprising a second applicator member disposed on the tubular member proximally of the balloon, the second applicator member being dimensioned to fit within the cervical canal in undilated condition, the lumen of the outer member having a diameter sufficient to permit passage of the second applicator member.

13. The cervical dilator of claim 12, wherein the second applicator member has a frustoconical body carrying one or more spirally disposed grooves or protrusions on its lateral surface.

14. The cervical dilator of claim 10, further comprising an inner member disposed between the tubular member and the outer member, the inner member having a proximal end and a distal end and a lumen therebetween, the tubular member reciprocating in the lumen of the inner member, the inner member reciprocating in the lumen of the outer member, a second applicator member being disposed at the proximal end of the inner member, the second applicator member being dimensioned to fit within the cervical canal in undilated condition, the lumen of the outer member having a diameter sufficient to permit passage of the second applicator member.

15. The cervical dilator of claim 14, wherein the second applicator member has a frustoconical body carrying one or more spirally disposed grooves or protrusions on its lateral surface.

16. A cervical dilator comprising:
an outer tubular member having a distal end, a proximal end, and a first lumen therebetween;
an inner elongated member having a distal end and a proximal end, a rounded cap being disposed at the distal end of the inner member and having a diameter larger than the diameter of the inner member, the inner member being configured to reciprocate within the first lumen and to extend beyond the distal end of the outer member, the inner member being made of a semi-rigid material and having a flexible wire attached longitudinally within the inner member;
an elongated balloon disposed distally on the outer member, the balloon having a size capable of dilating a cervical canal, the balloon being in fluid communication with a second lumen of the outer member.

17. The cervical dilator of claim 16, wherein the rounded cap is a sphere or an ellipsoid.

18. The cervical dilator of claim 16, wherein the balloon inflates to comprise a substantially cylindrical central portion and opposite proximal and distal tapered end portions.

19. The cervical dilator of claim 16, wherein a colored marking is positioned at the transition between the substantially cylindrical shape and the proximal tapered end portion.

20. The cervical dilator of claim 16, further comprising an injection device connected to the second lumen and dimensioned to progressively inflate the balloon.

21. The cervical dilator of claim 20, wherein the injection device inflates the balloon with a saline solution.

22. The cervical dilator of claim 20, wherein the injection device carries markings corresponding to the quantities of saline solution to be injected to cause a progressive inflation of one millimeter of the substantially cylindrical portion of the balloon.

23. The cervical dilator of claim 20, wherein the injection device is a syringe.

24. The cervical dilator of claim 16, wherein the inner member is has a tubular shape with an inner lumen, and wherein the flexible wire is attached to a wall of the inner lumen.

25. The cervical dilator of claim 16, wherein the inner member carries markings on its outer surface, enabling a clinician to measure the depth of insertion of the inner member into the cervical canal.

26. The cervical dilator of claim 16, wherein the outer tubular member has a distal portion and a proximal portion, wherein the distal portion has a larger diameter than the proximal portion, wherein the balloon is disposed on the distal portion, and wherein an applicator member is detachably disposed on the proximal portion.

27. The cervical dilator of claim 26, wherein the applicator member has a frustoconical body carrying one or more spirally disposed grooves or protrusions on its lateral surface, and wherein the applicator member is detachably disposed on the tubular member by detachably connecting the tubular member to a longitudinal lumen in the frustoconical body.

28. The cervical dilator of claim 27, wherein the longitudinal lumen in the frustoconical body has a diameter sufficiently large to enable the passage of the deflated balloon.

29. A method for dilating a cervical canal, the method comprising:
inserting a cervical dilator into the cervical canal, the cervical dilator including a tubular member having a distal end, a proximal end, and a lumen therebetween, the tubular member being made of a semi-rigid material, the cervical dilator further comprising a rounded cap disposed at the distal end and having a diameter larger than the diameter of the tubular member, a flexible wire extending in the lumen from the cap to the proximal end and attached to the lumen, a balloon being disposed distally on the tubular member, the balloon having a size capable of dilating the cervical canal, the lumen being adapted to be connected to an injection device at the proximal end;
measuring the depth of the uterus by progressively inserting the dilator into the uterus until contact of the rounded cap with a uterine wall;
retracting the dilator if necessary and adjusting the balloon within the cervical canal; and
inflating the balloon in subsequent diametral increments of about one millimeter by injecting a fluid from the injection device into the lumen and into the balloon.

30. The method of claim 29, wherein the balloon inflates to comprise a substantially cylindrical central portion and opposite proximal and distal tapered end portions, and wherein adjusting the balloon within the cervical canal includes positioning the proximal tapered end of the balloon to extend out of the cervical canal.

31. The method of claim 29, wherein inflating the balloon comprises injecting the balloon with a saline solution, and wherein injecting the balloon with a saline solution comprises providing the injection device with markings corresponding to the quantities of saline solution that cause a progressive inflation of the balloon of one millimeter.

32. The method of claim 29, further comprising the step of providing the tubular member with markings enabling a clinician to measure the depth of insertion of the tubular member into the cervical canal.

33. The method of claim 29, further comprising the step of anchoring a first applicator member to the cervical canal in dilated condition by providing an outer member having a proximal end and a distal end and a lumen therebetween, the tubular member reciprocating in the lumen of the outer member, the first applicator member being detachably disposed at the proximal end of the outer member, the first applicator member having a frustoconical body carrying one or more grooves or protrusions spirally disposed on its lateral surface, the first applicator member being dimensioned to fit within the cervical canal in dilated condition, the first applicator member being detachably disposed on the outer member by detachably connecting the tubular member to a longitudinal lumen in the frustoconical body, the first applicator member being inserted in the cervical canal after dilation.

34. The method of claim 33, further comprising the step of removing the tubular member with the balloon in deflated condition by detaching the outer member from the applicator member and by sliding the tubular member with the balloon in deflated condition through the longitudinal lumen in the frustoconical body.

35. The method of claim 33, further comprising the steps of:
anchoring a second applicator member to the cervical canal in undilated condition by providing the second applicator member disposed on the tubular member proximally of the balloon, the second applicator member being dimensioned to fit within the cervical canal in undilated condition, the second applicator member having a frustoconical body carrying one or more spirally disposed grooves or protrusions on its lateral surface, the lumen of the outer member having a diameter sufficient to permit passage of the second applicator member; and
removing the second applicator member from the cervical canal in dilated condition prior to anchoring the first applicator member.

36. The method of claim 33, further comprising the steps of:
anchoring a second applicator member to the cervical canal in undilated condition by providing the second applicator member disposed on an inner member disposed between the tubular member and the outer member, the inner member having a proximal end and a distal end and a lumen therebetween, the tubular member reciprocating in the lumen of the inner member, the inner member reciprocating in the lumen of the outer member, the second applicator member being disposed at the proximal end of the inner member, the second applicator member being dimensioned to fit within the cervical canal in undilated condition, the second applicator member having a frustoconical body carrying one or more spirally disposed grooves or protrusions on its lateral surface, the lumen of the outer member having a diameter sufficient to permit passage of the second applicator member; and
removing the second applicator member from the cervical canal in dilated condition prior to anchoring the first applicator member.

37. A method for dilating a cervical canal comprising:
inserting a cervical dilator into the cervical canal, the cervical dilator including an outer tubular member having a distal end, a proximal end, and a first lumen therebetween, the cervical dilator further comprising an inner elongated member made of a semi-rigid material and having a rounded cap disposed at the distal end, the rounded cap having a diameter larger than the diameter of the tubular member, the inner member being configured to reciprocate within the first lumen and to extend beyond the distal end of the outer member and into the uterine cavity, the inner member further having a flexible wire attached longitudinally within, the cervical dilator further comprising a balloon disposed distally on the outer member, the balloon having a size capable of dilating the cervical canal, the balloon being in fluid communication with a second lumen of the outer member, the second lumen being connected to an injection device;
measuring the depth of the uterus by progressively inserting the inner member into the uterus until contact of the rounded cap with a uterine wall;
retracting the inner member from the uterus and adjusting the balloon if necessary within the cervical canal; and
inflating the balloon in subsequent diametral increments of about 1 millimeter by injecting a fluid from the injection device into the lumen and into the balloon.

38. The method of claim 37, wherein the balloon inflates to comprise a substantially cylindrical central portion and opposite proximal and distal tapered end portions, and wherein adjusting the balloon within the cervical canal includes positioning the proximal tapered end of the balloon to extend out of the cervical canal.

39. The method of claim 38, wherein inflating the balloon comprises injecting the balloon with a saline solution, and wherein injecting the balloon with a saline solution comprises providing the injection device with markings corresponding to the quantities of saline solution that cause a progressive inflation of the balloon of one millimeter.

40. The method of claim 37, further comprising the step of providing the inner member with markings enabling a clinician to measure the depth of insertion of the tubular member into the cervical canal.

41. The method of claim 37, further comprising the step of anchoring an applicator member to the cervical canal by providing an applicator member detachably disposed at the proximal end of the outer member, wherein the applicator member has a frustoconical body carrying one or more spirally disposed grooves or protrusions on its lateral surface, and wherein the applicator member is detachably disposed on the outer member by detachably connecting the tubular member to a longitudinal lumen in the frustoconical body, and wherein the applicator member is inserted in the cervical canal.

42. The method of claim 41, further comprising the step of removing the outer and inner members from the cervical canal by detaching the outer member from the applicator member and by sliding the outer member with the balloon in deflated condition through the longitudinal lumen in the frustoconical body.
